(19) United States
(12) Patent Application Publication
(10) Pub. No.: US 2003/0086928 A1
(43) Pub. Date: May 8, 2003

(54) Compositions associated with complex formation and methods of use thereof

(76) Inventors: Marc Montminy, San Diego, CA (US); Brandee Wagner, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/247,279

(22) Filed: Sep. 17, 2002

Related U.S. Application Data
(63) Continuation-in-part of application No. PCT/US01/08946, filed on Mar. 19, 2001.
(60) Provisional application No. 60/190,705, filed on Mar. 17, 2000.

(51) Int. Cl. A61K 39/395; C07K 16/40

(52) U.S. Cl. 424/146.1; 530/388.26

(57) ABSTRACT

The present invention relates to compositions which specifically bind to a multimeric protein complex but not to the individual and separate components of the complex, or alternatively, compositions which specifically bind to individual and separate components but not to any complex thereof. The present invention also relates to methods of using such compositions for identifying compounds that promote multimeric complex formation and/or disruption and for treating disorders associated with aberrant complex formation by modulating the formation and/or disruption of multimeric protein complexes.

### RELATED APPLICATIONS

This application is a continuation-in-part of PCT Application Number PCT/US01/08946 entitled “COMPOSITIONS ASSOCIATED WITH COMPLEX FORMATION” and filed on Mar. 19, 2001, which claims priority to U.S. Provisional Patent Application No. 60/190,705, entitled “COMPOSITIONS ASSOCIATED WITH COMPLEX FORMATION AND METHODS OF USE THEREOF” and filed on Mar. 17, 2000.

### GOVERNMENTAL INTERESTS

This invention was made with Government support under Grant No. GM ROI 37828, awarded by the National Institutes of Health. The Government may have certain rights in the invention.

### FIELD OF THE INVENTION

The present invention relates to compositions specific for endogenous cellular protein-protein complexes or protein-nucleotide complexes, and methods of use thereof.

### BACKGROUND OF THE INVENTION

A number of signaling pathways propagate inductive signals via protein-protein interactions that are phosphorylation-dependent, including several second messenger pathways which modulate cellular gene expression via the phosphorylation of specific nuclear factors. The second messenger cAMP, for example, promotes target gene expression via the PKA mediated phosphorylation of a cyclic AMP response element binding protein (CREB) at Ser133 (Gonzalez and Montminy (1989) Cell 59:675-680). Although phosphorylation appears to enhance the nuclear import, multimerization, or DNA binding activities of certain factors, CREB belongs to a group of activators whose trans-activation potential is specifically affected (Brindle et al. (1993) Nature 364:821-824).

The CREB trans-activation domain is bipartite, consisting of constitutive and inducible activators that function synergistically in response to cAMP stimulation (Brindle et al. (1993); Quinn, P. G. (1993) J Biol Chem 268:16999-17009). The constitutive glutamine-rich activation domain, referred to as Q2, has been found to promote transcription via an interaction with transcription factor TFIID (Ferreri et al. (1994) Proc Natl Acad Sci USA 91:1210-1213).

By contrast, the kinase inducible domain (KID) stimulates target gene expression, following its phosphorylation at Ser133, by associating with the KIX domain of the co-activator CREB binding protein (CBP) (Arias et al. (1994) Nature 370:226-228; Chrivia et al. (1993) Nature 365:855-859; Kwok et al. (1994) Nature 370:223-226). The solution structure of the KID:KIX complex reveals that Ser133 phosphorylated KID undergoes a random coil to helix transition upon complex formation with KIX; and this transition in turn stabilizes the interaction between CREB and CBP (Radhakrishnan et al. (1997) Cell 91:741-752; Parker et al. (1998) Mol Cell 2:353-359).

In addition to cAMP, a wide variety of extra-cellular stimuli including phospho-inositol and calcium agonists, as well as certain growth factors such as NGF, EGF, IGF, and PDGF, appear to promote Ser133 phosphorylation of CREB with high stoichiometry (Brindle et al. (1995) Proc Natl. Acad Sci USA 92:10521-10525; Cesare et al. (1998) Proc Natl Acad Sci USA 95:12202-12207; Ginty et al. (1994) Cell 77:713-725; Pugazhenthi et al. (1999) J Biol Chem 274:2829-37; Seternes et al. (1999) Mol Endocrinol 13:1071-83). Yet these pathways are unable to promote target gene expression via CREB per se, reflecting either a block in recruitment of CBP/P300 or in the subsequent assembly of the transcriptional apparatus (Brindle et al. (1995)).

A number of methodologies, including co-immunoprecipitation (co-IP) and fluorescence resonance energy transfer (FRET), have been employed to evaluate protein-protein interactions (Zhou et al. (1998) Mol Endocrinol 12:1594-1604) (see also, Evans and Manjunatha, U.S. Pat. No. 5,928,896). Such procedures are limited by technical manipulations, such as protein extraction (co-IP) or over-expression (FRET), that may of themselves influence the recovery or detection of protein complexes.

Protein-protein interactions have been identified using complex specific compositions, such compositions consisting of antisera which characterized extracellular protein-protein interactions. For example, specific antisera have been described, most notably against HIV gp120 bound to its extracellular receptor CD4 (Kwonget al. (1998) Nature 393:648-659; Lee et al. (1997) J. Virol 87:6037-6043; DeVico et al. (1995) Virology 211:583-588). It was found that upon binding to CD4, gp120 appears to undergo a conformational change that exposes an epitope for recognition by complex specific antiserum.

A number of proteins appear to undergo structural changes upon complex formation with their cognate receptor or co-activator (Uesugi et al. (1997) Science 277:1310-1313). Clearly, there is a need for the development of complex-specific compounds, such as antisera, which may be generally useful for studies of cellular signaling.

### SUMMARY OF THE INVENTION

The present invention relates to compositions which specifically bind to a multimeric protein complex but not to the individual and separate components of the complex, or alternatively, compositions which specifically bind to individual and separate components but not to any complex thereof. The present invention also relates to methods of using such compositions for identifying compounds that promote multimeric complex formation and/or disruption and for treating disorders associated with aberrant complex formation by modulating the formation and/or disruption of multimeric protein complexes.

Some embodiments of the present invention relate to antibodies which recognize an epitope that is formed by the association of proteins in a multimeric complex. In some embodiments, the proteins that form the multimeric complex are unassociated prior to complex formation. In other embodiments, the proteins that form the multimeric complex are fused, linked or otherwise tethered to each other. In certain embodiments, the multimeric complex is formed by nuclear proteins. In some embodiments of the present invention, epitopes formed by the association of the components of the multimeric complex are created by the association of more than two proteins to form a multimeric complex. In other embodiments, the epitopes are created by the association of two proteins to form a dimeric complex.

In particular embodiments of the present invention, an antibody that specifically recognizes a conformational epitope specific for a dimeric protein complex is contemplated. In certain specific embodiments, the antibody recognizes a multimeric complex that is formed by the association of CREB and CBP. In other embodiments, the antibody binds to at least one amino acid residue in a KIX domain of CBP and/or binds to at least one amino acid residue in a KID domain of CREB. Upon formation of the CREB/CBP multimeric complex, the KID and/or KIX domains undergo a conformational change thereby facilitating the binding of the antibody. In some embodiments, the antibody that binds the CREB/CBP dimer is αKK.

In a related embodiment, the multimeric complex, which is recognized by the antibodies of the present invention, comprises a fusion protein. In some embodiments, the fusion protein comprises CREB coupled to CBP. In certain specific embodiments, the fusion protein comprises a KID domain of CREB coupled to a KIX domain of CBP. The coupling of proteins or protein domains to form the multimeric complex can be direct or mediated by a linker, such as a flexible or cleavable linker. In some embodiments, coupled proteins or protein domains are produced by genetic fusion. In other embodiments, coupled proteins or protein domains are produced by chemical linkage.

Some embodiments of the present invention relate to a method for identifying compositions specific for at least one component of a multimeric complex by providing a test solution containing at least one component of the multimeric complex and at least one candidate compound. The test solution is then contacted with an antibody that specifically recognizes a conformational epitope specific for the multimeric complex but does not bind to any individual component of the multimeric complex when the individual component is not part of the multimeric complex. The effect of the at least one candidate compound on the binding of the antibody to the multimeric complex is then determined. In some embodiments of the present invention, the multimeric complex that is formed comprises CREB and CBP. In certain embodiments, the multimeric complex formed in this method comprises a KID domain of CREB and a KIX domain of CBP. Alternatively, the multimeric complex may be formed by the coupling of CREB, or a domain thereof, to CBP, or a domain thereof, thereby forming a fusion protein. This coupling can be direct or mediated by a linker.

Another embodiment is a method for discovering candidate compounds that prevent the binding of an antibody described above to the CREB:CBP multimeric complex. In some embodiments, formation of the multimeric complex is prevented by the candidate compound, thereby preventing binding of an antibody specific to the multimeric complex. In still other embodiments, the candidate compound disrupts the multimeric complex, which also prevents the binding of the complex-specific antibody.

Some embodiments of the present invention relate to methods of administering an antibody specific for a multimeric protein complex as a treatment for diseases associated with aberrant complex formation.

### BRIEF DESCRIPTION OF THE DRAWINGS

FIG. 1 is a sequence comparison of homologous αA and (αB regions in the KID domains of CREB from Caenorhabditis elegans, CREM from mouse, and CREB from rat. Amino acid differences are bolded.

### DETAILED DESCRIPTION OF THE INVENTION

Embodiments of the present invention relate to compositions and methods for detecting or characterizing the interaction between intracellular (endogenous cellular) multimeric protein complexes, especially nuclear protein-protein complexes. In some embodiments, the compositions are antibodies that recognize a conformational epitope that is formed upon the association of two or more proteins to form a multimeric protein complex. By “conformational epitope” is meant an epitope that is created by the three-dimensional folding of a protein or association of proteins to form a protein complex. A change in how a protein folds may lead to the creation of one epitope and loss of another. In this way, the epitope is dependent on the conformation of the protein. Likewise a conformational epitope can be formed from the interaction of two or more proteins. For example, interactions which bring two or more proteins into close proximity can result in the formation of new conformational epitopes that are based on the association of specific three-dimensional domains from each protein. Such conformational epitopes would be disrupted upon dissociation of the protein complex.

The components of the multimeric protein complexes are either cytoplasmic, intracellular, or nuclear polypeptides, or derivatives thereof. Also included are cytoplasmic, intracellular or nuclear nucleic acids which interact with cytoplasmic, intracellular or nuclear polypeptides. A number of proteins form complexes by interacting with other endogenous cellular proteins. Those of skill in the art will recognize complexes which can be characterized by the present invention (see, e.g., Mayer B J. (1998) Methods Mol Biol. 84:33-48; Reeves W H. (1993), Mol Biol Rep. 17:153-4). In some embodiments, the individual components are nuclear factors such as nuclear receptors and co-activators, co-repressors, nucleic acid regulatory elements, and the like (see, e.g., Collingwood T N, et al. (1999) J Mol Endocrinol. 23:255-75, Manteuffel-Cymborowska M. (1999) Acta Biochim Pol. 46:77-89; Tenbaum S, et al. (1997) Int J Biochem Cell Biol. 29:1325-41, each incorporated herein by reference).

As referred herein, the term “complex” refers to a composition comprising two or more proteins, domains or fragments. The term also refers to the fusion of two or more proteins, domains or fragments which normally interact at the cellular level. For example, cyclic AMP response element binding protein (CREB) binds to CREB binding protein (CBP) intracellularly to activate gene transcription. A particular fusion protein contemplated by the present invention comprises the fusion of CREB and CBP, even more specifically, the domains KID and KIX from CREB and CBP, respectively. Various methods for production of such fusion proteins are well known in the art. The manipulations which result in their production can occur either at the gene or protein level. For example, the cloned coding region of the KID or KIX domains may be modified by any of numerous recombinant DNA methods known in the art (Sambrook et al., 1990, Molecular Cloning, A Laboratory Manual, 2d ed., Cold Spring Harbor Laboratory, Cold Spring Harbor, N.Y.; Ausubel et al., in Chapter 8 of Current Protocols in Molecular Biology, Greene Publishing Associates and Wiley Interscience, New York, the disclosures of which are incorporated herein by reference). It will be apparent from the following discussion that substitutions, deletions, insertions, or any combination thereof are introduced or combined to arrive at a final nucleotide sequence encoding the KID:KIX fusion protein.

Such fusion proteins may further comprise linkers or polypeptide spacers disposed between the various domains. In general, a linker is present between functional domains in a protein and has a function of linking the domains without affecting functions of the domains. “Linker” or “spacer” refers to a polypeptide sequence of about 3 to about 100 amino acids which allows the two proteins, domains or fragments to fold naturally on a consistent basis, i.e., in the shape consistent with that found in vivo. Such complexes, i.e., fusions, are extremely suitable to identify modulators of the independent components.

The protein, fragment or domain units can be independently oriented amino terminus to carboxy terminus within the complex protein, or vice versa. For example, the linker can be placed between the carboxy terminus of the first unit and the amino terminus of the second protein unit. Any type of linker known in the art can be used for linking the protein units in invention complex so long as the linker is flexible and does not interfere with dimerization between the units in the invention complex.

In one embodiment according to the present invention, the linker is a heterobifunctional cleavable cross-linker, such as N-succinimidyl (4-iodoacetyl)-aminobenzoate; sultosuccinimidyl(4-iodoacetyl)-aminobenzoate; 4-succinimidyi-oxycarbonyl-a-(2-pyridyldithia) toluene; sulfosuccinimidyl 1 a-methyl-a-(pyridyldithiol)-toluamidol hexanoate; N-succinimidyl 2-pyridyidithia)-proprionate; succinimidyl [3(-(pyridyldithio)-proprionamidol hexanoate; sulfosuccinimidyl [3(-2-pyridyldithio)-propionamidol hexanoate; 3-(2-pyridyldithio)-propionyl hydrazide, Ellman's reagent, dichlorotriazinic acid, S-(2-thiopyridyl)-L-cysteine, and the like. Further bifunctional linking compounds are disclosed in U.S. Pat. Nos. 5,349,066, 5,618,528, 4,569,789, 4,952,394, and 5,137,877, each of which is incorporated herein by reference in its entirety. These chemical linkers can be attached to purified proteins using numerous protocols known in the art, such as those described in Pierce Chemicals ‘Solutions, Cross-linking of Proteins: Basic Concepts and Strategies,” Seminar #12, Rockford, Ill.

In another embodiment according to the present invention, the linker can be a peptide having from about 2 to about 60 amino acid residues, for example from about 5 to about 40, or from about 10 to about 30 amino acid residues, such as is known in single-chain antibody research. Examples of such known linker moieties include GGGGS (SEQ ID NO.:1), GKSSGSGSESKS (SEQ ID NO.:2), GSTSGSGKSSEGKG (SEQ ID NO.:3), GSTSGSGKSSEGSGSTKG (SEQ ID NO.:4), GSTSGSGKPGSGEGSTKG (SEQ ID NO.:5), EGKSSGSGSESKEF (SEQ ID NO.:6), SRSSG (SEQ ID NO.:7), SGSSC (SEQ ID NO.:8), and the like. A Diphtheria toxin trypsin sensitive linker having the sequence AMGRSGGGCAGNRVGSSLSCGGLNLIAM (SEQ ID NO.: 9) is also useful. Additional linking moieties are described, for example, in Huston et al. (1988) PNAS 85:5879-5883; Whitlow et al. (1993) Protein Engineering 6:989-995; Newton et al. (1996) Biochemistry 35:545-553; A. J. Cumber et al. (1992) Bioconj Chem. 2:397-401; Ladurner et al. (1997) J. Mal. Hiol. M:330-337; and U.S. Patent. No. 4,894,443, each of which is incorporated herein by reference in its entirety.

Generally, the linker contains from about 5 to about 245 amino acids; however, there is no theoretical upper limit on the number of amino acids that could be used in the linker. In some embodiments, the linker contains from about 53 to about 125 amino acids. In other embodiments, the amino acids in the linker protein are selected to provide flexibility to the linker. In particular embodiments, a multiplicity of flexibility enhancing amino acids, such as proline, glycine, alanine and serine, are incorporated into the linker to enhance its flexibility.

Assuming a span of approximately 3.35 angstroms per amino acid within the flexible peptide bridge, the predicted minimum and maximum distance for the lengths of the linker having from 0 to 20 linker segments ranges from about 16.75 angstroms (the 5 amino acid bridge) to 804 angstroms (20-linker segments in addition to the 5 amino acid bridge). Thus, the length of the linker can readily be selected to enhance dimerization between any two particular members acting as partners by including as many linker segments as is preferred to enhance the biological functions of the functional dimer, as discussed herein.

One skilled in the art will appreciate that more than two proteins or domains can be coupled using the aforementioned linking techniques. For example, three, four, six, eight, and more then eight proteins may be coupled by using the linking techniques described above.

Under standard physiological conditions, the components of such multimeric complexes are capable of forming stable, non-covalent attachments with one or more of the other complex components. Methods for the purification and production of such multimeric complexes and the components thereof are well known to those skilled in the art (see, e.g., Margolis, B L, U.S. Pat. No. 6,037,134, issued Mar. 14, 2000, the disclosure of which is incorporated herein by reference in its entirety), as well as being described herein.

Protein-protein interactions are crucial to almost every physiological and pharmacological process. These interactions often are characterized by very high affinity, with dissociation constants in the low nanomolar to subpicomolar range. Such strong affinity between proteins is possible when a high level of specificity allows subtle discrimination among closely related structures. The interaction sites of several protein pairs have been identified by strategies such as chemical modification of specific amino acid residues, site-directed mutagenesis, peptide synthesis, X-ray diffraction studies and theoretical approaches.

Certain general structural features have emerged from these studies. For example, some interactions involve more than one interaction site. The phrase “interaction” is used herein to denote a site or domain comprised of amino acid residues which is involved in the connection between two proteins, whether it be physical, chemical or otherwise. Moreover, an interaction site or domain can be comprised of one or more amino acids that respond to an effect that is generated by one or more other protein domains or amino acid regions. The high affinities at these interaction sites are attributed to several factors, including but not limited to, shape complementarity, electrostatic and hydrogen bond links, and burial or interaction of hydrophobic groups. A protein-protein interaction may involve one or more of these factors at each interaction site.

The amino acids of an interaction site usually constitute a small proportion of the total amino acids present in the polypeptide. Typically, the number of amino acid residues in a single interaction site ranges from three to six. These residues often are connected by the peptide bonds of adjacent residues in a continuous interaction site. Alternatively, the amino acid residues involved in the interaction are not linked directly by peptide bonds, but rather are brought together by the three-dimensional folding of the protein and are known as “discontinuous” sites. Due to this extensive variability, it has been difficult to identify the amino acids of interaction sites.

The chemical nature of the side chains of the amino acid residues contributes significantly to the interaction, although main chain atoms also can be involved. Positively charged residues (such as lysine, arginine and histidine) can associate through salt bridge links with negatively charged residues (such as aspartic acid and glutamic acid). Additionally, the side chains of leucine, isoleucine, methionine, valine, phenylalanine, tyrosine, tryptophan and proline are often involved in hydrophobic interactions. Precise alignment of atoms between the interaction sites of one protein and its partner also allow multiple Van der Waals interactions and thus increase the likelihood of strong binding between the two interaction partners.

Specifically, the embodiments described herein illustrate a novel approach to the study of cellular signaling. For example, αKK antiserum binds in part to residues in KID that undergo a conformational change following complex formation with KIX. The ability of αKK antiserum to recognize full-length CREB:CBP complexes strongly supports the notion that a helical transition also occurs within the context of the full length CREB protein. Structural transitions in transcription activators like CREB may therefore be integral to the process of recruiting the transcriptional machinery.

CREB:CBP complexes appear to be formed at discrete regions within the nucleus. Although the constituents of these complexes are unknown aside from CREB and CBP, they may contain other components of the transcriptional apparatus. In this regard, CBP has been found to associate with RNA polymerase II holoenzyme complexes (Nakajima et al. (1997) Genes Dev 11:738-747; McKenna et al. (1998) Proc Natl Acad Sci USA 95:11697-11702; Cho et al. (1998) Mol Cell Bio 18:5355-5363) as well as PML-containing nuclear bodies (LaMorte et al. (1998) Proc Natl Acad Sci USA 95: 4991-4996).

The ability of αKK antiserum to distinguish between different signaling pathways demonstrates the utility of this reagent in monitoring cellular activity compared to phospho (Ser133) CREB antiserum. Phospho-CREB specific antisera have been widely used, particularly in neuronal cells, to evaluate cellular responses to various stimuli. This data suggests that some subset of these signals may not elicit a transcriptional response, at least via the same pathway as cAMP.

Phosphorylation of CREB in response to TPA is likely to be indirect, possibly involving ERK1,2 and PP90_{RSK}. Activation of the MAPK pathway may inhibit CREB/CBP complex formation by inducing phosphorylation of CREB at other inhibitory sites. In this regard, phosphorylation of CREB at Ser142 has been shown to block target gene activation, in part, by blocking CREB/CBP complex formation (Parker et al. (1998); Sun et al. (1994) Genes Dev 8:2527-2539). The compositions disclosed herein can be utilized to determine the mechanism by which CREB discriminates between cAMP and other second messenger pathways.

In light of the present disclosure, one of ordinary skill in the art is enabled to practice novel methods which are useful in the identification of proteins and other compounds which specifically bind to, or otherwise directly interact with, the complex or with each individual and separate component. In general, methods for identifying compositions specific to the complex but not the individual and separate components are contemplated. Compositions that bind to only the protein complex are screened and isolated by identifying all compounds which bind to the complex, and thereafter removing those compounds which also bind the individual and separate components. By removing the component-specific compositions, the remaining compositions are complex-specific and can be utilized to detect complex formation and/or induce complex formation and/or activity by increasing complex stability. Alternatively, compositions are screened and isolated by identifying compounds which bind to at least one of the components but not to the complex. Such compositions can be produced by identifying compounds which bind to the individual and separate components, and thereafter removing those compounds which bind or interact with the complex. By screening for component-specific compositions, such compositions can be employed to characterize the interaction domains (of the components not normally accessible when in a complex) and/or inhibit or decrease complex formation and/or activity.

Depending on the complex involved, enhancing or inhibiting the interactions between component members may have differing modulatory effects on subsequent signal transduction. “Formation”, as used herein, refers not only to physical cooperation of complex components, but also to synergism of the activity of the complexes, regardless of whether or not such complexes remain able, physically, to form. Contrarily, “disruption”, as used here, is meant to refer not only to a physical separation of complex components, but also refers to a perturbation of the activity of the complexes, regardless of whether or not such complexes remain able, physically, to form. “Activity,” as used herein, refers to the function of the complex in the signal transduction cascade of the cell in which such a complex is formed, e.g., activity refers to the function of the complex in effecting, enhancing or inhibiting cellular signaling, transcription, and the like. For example, the effect of complex formation and/or disruption may augment, reduce, or block the signal normally transduced into the cell. In embodiments where the compositions described herein are used in the treatment of disorders resulting from aberrant complex formation, augmentation or reduction of the signal normally transduced into the cell may be desirable for the treatment depending on the particular disorder. As used herein, “aberrant multimeric complex formation” or “aberrant complex formation” means improper association of complex components or improper complex activity which results in a disorder. This term also encompasses the lack of association of complex components so as to result in a disorder.

Because the normal physiological roles of complex formation and/or disruption for a number of protein-protein interactions are still unknown, compounds which bind to complexes or the individual and separate components thereof have utility in treatments and diagnostics. Compounds which bind only to complexes can act to facilitate characterization of complex formation, detect complex formation or enhance the normal activity of the complex. Such compounds can also be used to compensate for lost or abnormal activity of mutant forms of the complex wherein one or all of the components of the complex are mutants. Alternatively, compounds which only bind to the individual and separate components can facilitate the identification of the interaction domains of each component as well as inhibit formation of the complex thereby modulating complex activity.

The effect of agents which bind to the complex or components thereof can be monitored either by the direct monitoring of this binding using instruments (e.g., BIAcore, LKB Pharmacia, Sweden) to detect this binding by, for example, a change in fluorescence, molecular weight, or concentration of either the binding agent, complex or components, either in a soluble phase or in a substrate-bound phase.

Methods for screening cellular lysates, tissue homogenates, or small molecule libraries for candidate complex-specific or component-specific binding compounds are well known in the art and, in light of the present disclosure, can be employed to identify compounds which bind specifically to the complex or the individual and separate components or which modulate complex activity as defined by non-specific measures (e.g., changes in intracellular signaling, transcription, and the like) or by specific measures (e.g., changes in downstream peptide production or changes in the expression of other downstream genes which can be monitored by differential display, two-dimensional gel electrophoresis, differential hybridization, or serial analysis of gene expression (SAGE) methods). The specific embodiments involve variations on the following techniques: (1) direct extraction by affinity chromatography; (2) immunocytochemical experiments; (3) the Biomolecular Interaction Assay (BIAcore); (4) the yeast two-hybrid systems, and the like. As will be appreciated by one of ordinary skill in the art, there are numerous other methods of screening individual proteins or other compounds, as well as large libraries of proteins or other compounds (e.g., phage display libraries and cloning systems from Stratagene, La Jolla, Calif.) to identify molecules which specifically bind to the complex or the components. For example, some methods generally combine the steps of mixing either the complex or component(s) (fusion or fragment) with test compounds, allowing for binding (if any), and assaying for bound complexes.

The compositions of some embodiments of the present invention include endogenous cellular components which interact with the complexes or components in vivo and which, therefore, provide new targets for pharmaceutical and therapeutic interventions, as well as recombinant, synthetic and otherwise exogenous compounds which may have complex or component binding capacity and, therefore, may be candidates for pharmaceutical agents. Thus, in one series of embodiments, cell lysates or tissue homogenates may be screened for proteins or other compounds which specifically bind to either the complex or components thereof. In addition, immunogenic compositions can be employed to distinguish epitopes specific to the complex, as well as epitopes specific to the components. Alternatively, any of a variety of exogenous compounds, both naturally occurring and/or synthetic (e.g., libraries of small molecules or peptides), may be screened for complex-specific or component-specific binding capacity.

Once identified by the methods described above, the candidate compounds may then be produced in quantities sufficient for pharmaceutical administration or testing (e.g., microgram, milligram or greater quantities), and formulated in a pharmaceutically acceptable carrier (see, e.g., Remington's Pharmaceutical Sciences, Gennaro, A., ed., Mack Pub., 1990).

In addition, once identified by the methods described above, the candidate compounds may also serve as “lead compounds” in the design and development of new pharmaceuticals, e.g., design and development of pharmaceuticals which enhance or inhibit complex formation (see, e.g., Farber G K (1999) Pharmacol Ther. 84:327-32, the disclosure of which is incorporated herein by reference in its entirety). For example, as in well known in the art, sequential modification of small molecules (e.g., amino acid residue replacement with peptides; functional group replacement with peptide or non-peptide compounds) is a standard approach in the pharmaceutical industry for the development of new pharmaceuticals. Such development generally proceeds from a “lead compound” which is shown to have at least some of the activity (e.g., complex-specific or component-specific binding or blocking ability) of the desired pharmaceutical.

The compositions or other compounds identified by these methods may be purified and characterized by any of the standard methods known in the art. Proteins may, for example, be purified and separated using electrophoretic (e.g., SDS-PAGE, two-dimensional PAGE) or chromatographic (e.g., HPLC) techniques and may then be microsequenced. For proteins with a blocked N-terminus, cleavage (e.g., by CNBr and/or trypsin) of the particular binding protein is used to release peptide fragments. Further purification and/or characterization by HPLC and microsequencing and/or mass spectrometry by conventional methods provide internal sequence data on such blocked proteins. For non-protein compounds, standard organic chemical analysis techniques (e.g., IR, NMR and mass spectrometry; functional group analysis; X-ray crystallography) may be employed to determine their structure and identity.

Agents which act intracellularly to modulate the formation and/or disruption of the multimeric complexes described herein may be small organic or inorganic compounds. Examples of such molecules can be found, for example, in Schreiber S. L. (2000) Science 287:1964-1969; Seymour L. (1999) Cancer Treat Rev 25:301-12; Mendonca et al. (1999) Med Res Rev 19:451-62; the disclosures of each incorporated herein by reference in their entireties). Small molecules are particularly useful in the intracellular context because they are more readily absorbed after oral administration, have fewer potential antigenic determinants, and/or are more likely to cross lipid and/or nuclear membranes than larger molecules such as nucleic acids or proteins. The methods described herein can be used to identify these small molecule modulators of complex formation.

Alternatively, antibodies capable of binding an epitope specific to a multimeric complex are contemplated for use in modulating the formation of such complexes as well as for the diagnosis and treatment of disorders involving aberrant complex formation, e.g., cancers, and the like.

Described herein are antibodies and methods for the production of antibodies which are capable of specifically recognizing a complex of intracellular proteins or an epitope thereof. In some embodiments, antibodies that recognize the multimeric complex but not an individual component of the complex when each individual component is present separate and apart from the complex are described. Such antibodies may include, but are not limited to polyclonal antibodies, monoclonal antibodies (mAbs), humanized or chimeric antibodies, single chain antibodies, Fab fragments, F(ab′)₂ fragments, fragments produced by a FAb expression library, anti-idiotypic (anti-Id) antibodies, and epitope-binding fragments of any of the above. Such antibodies may be used, for example, in the detection of a complex in a biological sample, or, alternatively, as a method for enhancing complex formation, thus, increasing complex activity. As used herein, an antibody that “recognizes” a multimeric complex or an epitope thereof means an antibody capable of binding to the multimeric complex or an epitope thereof.

Alternatively, described herein are antibodies and methods for the production of antibodies which are capable of specifically recognizing an epitope on either the components of the complex, especially epitopes on each individual and separate component which would not be recognized by an antibody which would bind the complex. Such antibodies may include, but are not limited to polyclonal antibodies, monoclonal antibodies (mAbs), humanized or chimeric antibodies, single chain antibodies, Fab fragments, F(ab′)₂ fragments, fragments produced by a FAb expression library, anti-idiotypic (anti-Id) antibodies, and epitope-binding fragments of any of the above. Such antibodies may be used, for example, in the detection of a complex in a biological sample, in the identification and characterization of the interaction domains of the components of a complex or, alternatively, as a method for the inhibition of complex formation, thus, decreasing complex activity. As used herein, an antibody that “recognizes” a component of a multimeric complex or an epitope of such component means an antibody capable of binding to the component of the multimeric complex or an epitope of such complex.

Immunogen preparations of complexes or complex components can be produced from crude extracts (e.g., membrane fractions of cells highly expressing the complex or components), from portions of the complex or peptides substantially purified from cells which naturally or recombinantly express them or, in the case of small immunogens, by chemical peptide synthesis.

Polyclonal antibodies are heterogeneous populations of antibody molecules derived from the sera of animals immunized with an antigen, such as the multimeric complex, the individual and separate components of the complex, or antigenic functional derivatives thereof. For the production of polyclonal antibodies, various host animals may be immunized by injection with the multimeric complex or components thereof including but not limited to rabbits, mice, rats, etc. Various adjuvants may be used to increase the immunological response, depending on the host species, including but not limited to Freund's (complete and incomplete), mineral gels such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

A monoclonal antibody, which is a substantially homogeneous population of antibodies to a particular antigen, may be obtained by any technique which provides for the production of antibody molecules by continuous cell lines in culture. These include, but are not limited to the hybridoma technique of Kohler and Milstein (1975) Nature 256:495-497; and U.S. Pat. No. 4,376,110), the human B-cell hybridoma technique (Kosbor et al. (1983) Immunology Today 4:72; Cole et al. (1983) Proc. Natl. Acad. Sci. USA 80:2026-2030), and the EBV-hybridoma technique (Cole et al. (1985) “Monoclonal Antibodies And Cancer Therapy,” Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al. (1984) Proc. Natl. Acad. Sci. 81:6851-6855; Neuberger et al. (1984) Nature 312:604-608; Takeda et al. (1985) Nature 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird (1988) Science 242:423-426; Huston et al. (1988) Proc. Natl. Acad. Sci. USA 85:5879-5883; and Ward et al. (1989) Nature 334:544-546) can be adapted to produce complex-specific or components-specific single chain antibodies. Single chain antibodies are formed by linking the heavy and light chain fragment of the Fv region via an amino acid bridge, resulting in a single chain polypeptide.

Antibody fragments which contain specific binding sites for a multimeric complex or components thereof may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al. (1989) Science 246:1275-1281) to allow rapid and easy identification of monoclonal Fab fragments with the desired specificity to the multimeric complex or components thereof.

One embodiment of the present invention is an immunoassay to detect the formation of a multimeric complex in a sample. The immunoassay comprises at least one monoclonal antibody that preferentially or exclusively binds a complex. Alternatively, the immunoassay may comprise two monoclonal antibodies. In this embodiment, the first monoclonal antibody may be an anti-complex monoclonal antibody and the second monoclonal antibody may be an anti-component(s) monoclonal antibody, or another anti-complex monoclonal antibody. One or more of the monoclonal antibodies may be labeled. Some assays comprise an enzymatically labeled monoclonal antibody.

The immunoassay described above can also utilize a monoclonal antibody which is immobilized on a solid support. The solid support may be composed, for example, of materials such as glass, paper, polystyrene, polypropylene, polyethylene, dextran, nylon, amylases, natural and modified celluloses, polyacrylamides, agaroses, or magnetite. The nature of the support can be either soluble to some extent or insoluble for the purpose of the present invention. The support material may have virtually any possible structural configuration. Thus, the support configuration may be spherical, as in a bead, or cylindrical, as in the inside surface of a test tube, or the external surface of a rod. Alternatively, the surface may be flat, such as a sheet, test strip, etc. Those skilled in the art will appreciate many other suitable carriers for binding monoclonal antibody, or will be able to ascertain the same by use of routine experimentation. In one embodiment, the support will be a polystyrene microtiter plate.

Numerous immunoassay formats and procedures are known in the art. Conventional radioimmunoassay (RIA) procedures, for example, were described by Yalow et al. (1990)J. Clin. Invest. 39:1157. The immunoassay of the present invention can be in any format, although a preferred immunoassay utilizes an enzymatic microtiter plate (MP) immunoassay format.

The immunoassay may be enhanced by several means, including the addition of detergent, for example, NP-40, to the assay incubation buffer. Addition of NP-40 to the immunoassay has been found to beneficially reduce the non-specific binding, especially of ACT and its complexes in serum. The amount of NP-40 added to the immunoassay is sufficient to reduce the non-specific binding, with a preferred embodiment using a concentration of NP-40 of about 0.4%.

Non-specific binding can also be reduced by the addition of microparticles to the immunoassay. The microparticles are preferably made of latex. Any size microparticles may be used which reduce the non-specific binding, however, most preferably, latex microparticles of approximately 0.088 micron are used. The concentration of microparticles is sufficient to beneficially reduce non-specific binding. In a preferred embodiment, a concentration of latex microparticles of approximately 0.1% is used.

The antibodies described herein can be bound to many different carriers and used to detect the presence of an epitope present only on a multimeric complex or an epitope associated with a complex component. Examples of well known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to the antibody, or will be able to ascertain such, using routine experimentation.

In using the monoclonal and polyclonal antibodies of the invention for the in vivo detection of antigen, e.g., complex formation or inhibition, the detectably labeled antibody is given a dose which is diagnostically effective. The term “diagnostically effective” means that the amount of detectably labeled antibody is administered in sufficient quantity to enable detection of the antigen for which the antibodies are specific.

The concentration of detectably labeled antibody which is administered should be sufficient such that the binding to complexes in cells is detectable compared to the background. Further, it is desirable that the detectably labeled antibody be rapidly cleared from the circulatory system in order to give the best target-to-background signal ratio.

As a rule, the dosage of detectably labeled antibody for in vivo diagnosis will vary depending on such factors as age, sex, and extent of disease of the individual. Such dosages may vary, for example, depending on whether multiple injections are given, antigenic burden, and other factors known to those of skill in the art. The dosage of monoclonal antibody can vary from about 0.001 mg/m² to about 500 mg/m², preferably 0.1 mg/m² to about 200 mg/m², most preferably about 0.1 mg/m² to about 10 mg/m². Such dosages may vary, for example, depending on whether multiple injections are given, and other factors known to those of skill in the art.

In using a monoclonal antibody for the in vivo detection of antigen, the detectably labeled monoclonal antibody is given in a dose which is diagnostically effective. The term “diagnostically effective” means that the amount of detectably labeled monoclonal antibody is administered in sufficient quantity to enable detection of the antigen for which the monoclonal antibodies are specific. The concentration of detectably labeled monoclonal antibody which is administered should be sufficient such that the binding to complexes in cells is detectable compared to the background. Further, it is desirable that the detectably labeled monoclonal antibody be rapidly cleared from the circulatory system in order to give the best target-to-background signal ratio.

For in vivo diagnostic imaging, the type of detection instrument available is a major factor in selecting a given radioisotope. The radioisotope chosen must have a type of decay which is detectable for a given type of instrument. Still another important factor in selecting a radioisotope for in vivo diagnosis is that the half-life of the radioisotope be long enough so that it is still detectable at the time of maximum uptake by the target, but short enough so that deleterious radiation with respect to the host is minimized. Ideally, a radioisotope used for in vivo imaging will lack a particle emission, but produce a large number of protons in the 140-250 keV range, which may be readily detected by conventional gamma cameras.

For in vivo diagnosis, radioisotopes may be bound to immunoglobulin either directly or indirectly by using an intermediate functional group. Intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions which can be bound to the monoclonal antibodies of the invention are 111 In, 97 Ru, 67 Ga, 68 Ga, 72 As, 89 Zr, and 201T1.

A monoclonal antibody useful in the method of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include 157Gd, 55 Mn, 162 Dy, 52Cr, and 56 Fe.

In another series of embodiments, the present invention provides methods for diagnosing victims of disorders associated with aberrant complex formation. Diagnosis can be accomplished by methods based upon the compositions described or identified herein. In accordance with these embodiments, diagnostic kits are also provided which will include the reagents necessary for the above-described diagnostic screens.

In another series of embodiments, the present invention provides methods and pharmaceutical preparations for use in the treatment of disorders associated with aberrant complex formation, e.g., cancers, and the like. Also contemplated are disorders associated with metabolism, cellular signaling, and the like. Such pharmaceutical preparations are based upon compositions which disrupt or promote the formation of the multimeric complex, including antibodies which recognize the complex or an epitope specific to the complex but which do not recognize individual complex components. Alternatively, pharmaceuticals may be based upon antibodies which recognize individual complex components but not the multimeric complex. Pharmaceutical preparations of small molecules (drugs) which modulate complex formation or disruption by altering the structure or activity of the complex or component are also contemplated. For example, compositions specific to the complex can increase complex formation by increasing complex stability and/or complex-specific conformation (see, e.g., Wu H, et al. (1999) Thromb Res 95:245-53).

Some embodiments of the present invention provide methods of treating individuals having a disorder resulting from aberrant formation of a multimeric complex. To implement these methods, an antibody or other compound that disrupts or promotes the formation of a multimeric protein complex can be formulated in a pharmaceutically acceptable carrier using methods well known in the art. For the treatment of a disorder resulting from the lack of association of the components of a multimeric protein complex, antibodies or compounds which promote association of the complex components are selected for administration to the individual having the disorder. For the treatment of a disorder resulting from irregular association of the components of a multimeric protein complex, antibodies or compounds which disrupt the association of the complex components are selected for administration to the individual having the disorder.

As a rule, the dosage of detectably labeled antibody for treatment will vary depending on such factors as age, sex, and extent of disease of the individual. Such dosages may vary, for example, depending on whether multiple injections are given, antigenic burden, and other factors known to those of skill in the art. The dosage of monoclonal antibody can vary from about 0.001 mg/m² to about 500 mg/m², preferably 0.1 mg/m² to about 200 mg/m², most preferably about 0.1 mg/m² to about 10 mg/m². Such dosages may vary, for example, depending on whether multiple injections are given, and other factors known to those of skill in the art.

The following examples are intended to illustrate but not to limit the invention in any manner, shape, or form, either explicitly or implicitly. While they are typical of those that might be used, other procedures, methodologies, or techniques known to those skilled in the art may alternatively be used.

### EXAMPLES

Here, the present examples characterize novel compounds that specifically bind to the CREB:CBP complex, but do not bind to either protein individually. Specifically, a novel complex specific antiserum was employed to monitor the CREB:CBP interaction following exposure to various stimuli. Epitope mapping experiments demonstrated that the CREB:CBP antiserum detects residues in KID that undergo a conformational change upon binding to KIX. The ability of this antiserum to recognize full length CREB:CBP complexes in a phospho (Ser133) dependent manner demonstrated that the structural transition observed with the isolated KID domain also occurs in the context of the full length CREB protein. Immunocytochemical experiments revealed that CREB:CBP complex formation, in response to cAMP, is limited to discrete compartments within the nucleus. Remarkably, other stimuli were found to have distinct effects on complex formation, even in light of comparable potency at the level of Ser133 phosphorylation.

### Example 1

### Experimental Procedures

Preparation of CREB:CBP Specific Antiserum

KID and KIX were expressed in E.coli BL21 cells and purified as previously described (Radhakrishnan et al. (1997)). The peptides were crosslinked with glutaraldehyde in crosslinking buffer (20 mM Hepes pH 7.5, 100 mM KCl, 2 mM MgCl₂ and 2 mM EDTA). For αKK antiserum, IgG was purified by 50% ammonium sulfate precipitation followed by protein A agarose affinity purification. Antibodies to phospho-KID or KIX were separately pre-absorbed by incubation with a phospho-KID and KIX coupled Affi-gel 10 resins. KID/KIX complex specific antibodies were then purified by incubating with a phospho-KID/KIX coupled Affi-gel 10 resin and eluting with 100 mM glycine. For CREB/p300 co-immunoprecipitations, 100 ng of recombinant p300 (gift from P. Nakatani) and 2 μg of recombinant CREB or phospho(Ser133) CREB were co-incubated, and the immunoprecipitates were processed as previously described (Kee et al. (1996) J. of Biol Chem 271:2373-2375, the disclosure of which is incorporated herein by reference in its entirety). Gel shift assays with ³²P-labeled CRE or GAL4-RE oligonucleotides were performed as reported (Gonzalez and Montminy (1989) Cell 59:675-680, the disclosure of which is incorporated herein by reference in its entirety).

Immunocytochemistry

D5 cells were grown on glass coverslips and stimulated with forskolin and 3-isobutyl-1-methylxanthine (IBMX) or 12-O-tetradecanoyl phorbol acetate (TPA) for 10 minutes. The cells were methanol-fixed at 10° C. for 5 min followed by three 5 min washes in PBS. Cells were blocked for 30 min with 3% BSA in PBS and donkey serum diluted 1:50. Primary antibodies were diluted in 3% BSA/PBS to 1:2000 for the phospho-CREB specific antibody 5322 or 1:100 (3 μg/ml) for the KID/KIX specific antibody. The cells were incubated with the primary antibodies for 1 hr at RT, followed by 3 washes with PBS and a 1 hr incubation with Biotin-SP-conjugated Donkey anti rabbit IgG diluted 1:200 in 3% BSA/PBS. After 3 washes in PBS, Texas Red conjugated Streptavidin was added at 1:200 dilution in 3% BSA/PBS. After one hour incubation, cells were washed 3 times in PBS and mounted in 90% glycerol/PBS containing 1 mg/ml phenylenediamine.

### Example 2

### Evaluation of Complex Formation in vivo

To evaluate CREB:CBP complex formation in vivo, we developed a complex specific antiserum using glutaraldehyde cross-linked phospho(Ser133) KID:KIX complexes as immunogen.

Recombinant phospho (Ser133) KID and KIX peptides from CREB and CBP, respectively, were cross-linked with glutaraldehyde and then employed as immunogen to generate KID:KIX specific antisera. Anti-KID/KIX (αKK) antiserum was initially purified from crude serum of immunized rabbits by chromatography over separate KID and KIX resins to remove antibodies that could recognize either phospho (Ser133) KID or KIX peptides independently. Flow-through fractions from these columns were then passed over resin containing cross-linked KID:KIX peptides, and the bound antibody fraction, referred to as αKK antiserum, was acid eluted.

Western blot assays of phospho (Ser133) KID, KIX, and phospho (Ser133) KID:KIX complexes were assayed using affinity purified KID:KIX specific antiserum (αKK). In Western blot assays, αKK antiserum could recognize cross-linked KID KIX complexes but not KIX or phospho (Ser133) KID peptides alone. Western blot assay was also performed with anti-P300 antiserum. P300 was recovered from αKK immunoprecipitates following incubation of antiserum with purified full length P300, P300 plus unphosphorylated CREB, or P300 plus phospho (Ser133) CREB. Fifty percent of total P300 protein was added to each immunoprecipitation reaction. Consistent with the notion that αKK antiserum is also competent to detect complex formation between full-length phospho(Ser133)CREB and CBP/P300 proteins, P300 was recovered from immunoprecipitates of recombinant P300 and phospho(Ser133)CREB but not of P300 plus unphosphorylated CREB. αKK antiserum also detected the phospho (Ser133) dependent recruitment of CBP in immunoprecipitation assays, demonstrating the capacity of this antiserum to recognize complexes formed with both co-activator using autoradiagram of in vitro translated ³⁵S-CBP following co-incubation with (αKK alone, with unphosphorylated CREM, or PKA-phosphorylated CREM.

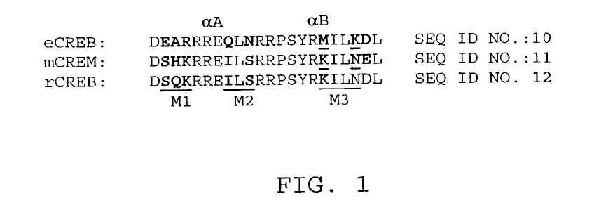

The KID domain is highly conserved amongst CREB family members, particularly in residues that function in protein-protein interactions with KIX (Radhakrishnan et al. (1997)). In gel mobility shift assays, for example, αKK antiserum was also capable of binding to KIX complexes formed with the mammalian CREB homolog CREM but not with the more distantly related C. elegans CREB polypeptide (eCREB). Gel mobility shift assays were prepared with C. elegans phospho (Ser54) CREB (eP-CREB) and murine phospho (Ser71) CREM using a ³²P-labeled CRE oligonucleotide. Reactions contained either eCREB or CREM plus KIX, αKK antiserum, or α-phospho-specific CREB antiserum. Compared to its mammalian counterpart, eCREB contains a number of amino acid substitutions within its KID domain (FIG. 1), prompting evaluation of whether these constituted an important epitope for αKK recognition.

### Example 3

### Gel Mobility Shifts

Gel mobility shift assays of wild-type and mutant (M1, M2, M3) GAL4-KID polypeptides using double stranded GAL4 binding site oligonucleotide were performed to determine the relative migration of GAL4-KID (P-KID), GAL4-KID:KIX (pKID:KIX) and αKK supershifted (Ab:KID:KIX) complexes bound to the ³²P-labeled GAL4 oligonucleotide. With reference to FIG. 1, mutation of the residues indicated in region M1 in the αA helix of the KID domain of rCREB to the corresponding amino acids of eCREB had no effect either on complex formation with KIX or on antibody recognition by αKK by gel shift assay. Mutation of the amino acid residues indicated in region M2 or those indicated in region M3 of the KID domain partially disrupted interaction with KIX by gel shift assay. Although these residues do not appear to form surface contacts with KIX (Radhakrishnan et al. (1997)), mutation at these amino acids may impose structural constraints on the mutant KID peptides that make complex formation less favorable. Nevertheless, complexes formed with mutant M2 KID were supershifted by αKK antiserum; but complexes formed with mutant M3 KID, which contained a lysine to methionine substitution at position 136 and an asparagine to lysine substitution at position 139 in the αB region, were not. Taken together, these results indicate that residues Lys136 and Asn139 are critical for recognition by αKK antiserum.

Lys136 and Asn139 are directly aligned on the solvent face of helix αB, a region in KID that undergoes a random coil to helix transition upon complex formation with KIX. The importance of these residues for recognition by αKK suggests that the antiserum detects, in part, the conformational change in KID that accompanies complex formation with KIX. Moreover, the ability of αKK to recognize full-length CREB:CBP complexes suggests that the structural change detected by NMR analysis with KID and KIX peptides, also occurs in the context of the full length proteins.

In addition to cAMP, other stimuli such as the phorbol ester TPA can promote Ser133 phosphorylation of CREB; yet these stimuli are unable to induce target gene activation via CBP, reflecting either a block in CREB:CBP complex formation or in the subsequent recruitment of the transcriptional apparatus. To evaluate formation of CREB:CBP complexes in vivo, NIH 3T3 cells expressing chromosomal copies of the rat somatostatin gene, hereafter referred to as D5 cells (Montminy et al. (1986) J Neurosci 6:803-813) were used. Treating D5 cells with TPA induced Ser133 phosphorylation of CREB with comparable stoichiometry to forskolin induction when analyzed by Western blot assay with phospho-specific CREB antiserum 5322. Western blot assays of total CREB and phospho (Ser133) CREB (P-CREB) levels were performed in control (C) or treated D5 cells exposed to either forskolin (F) or TPA (T) for 30 minutes. Northern blot assays were then performed to detect somatostatin (SOM) and tubulin (TUB) mRNA levels in control D5 cells (C) and in D5 cells treated with forskolin (F) or TPA (T) for 4 hours. Forskolin stimulated somatostatin mRNA accumulated 5-fold in D5 cells, whereas TPA had no discernible effect.

Consistent with the absence of phospho (Ser133), CREB staining under basal conditions in untreated D5 cells by immunofluorescence analysis with αKK antiserum revealed no CREB:CBP complexes. D5 cells were then treated with forskolin or TPA for 10 minutes. Treatment with forskolin induced accumulation of phospho (Ser133) CREB and correspondingly promoted the appearance of CREB:CBP complexes. By contrast with forskolin, however, no CREB:CBP complexes were detected in TPA-treated cells despite comparable levels of Ser133 phosphorylation.

### Example 4

### Confirmation of Complex Specificity

To confirm the specificity of the αKK antiserum, Immunostaining of forskolin treated (10 μM, 10 min) CREB−/− and CREB+/+ fibroblasts from knockout and wild type littermate mice were employed (Rudolph et al. (1998) Proc Natl Acad Sci USA 95:4481-4486, the disclosure of which is incorporated herein by reference in its entirety). Compared with cells from wild-type littermates, which showed abundant nuclear staining following treatment with cAMP agonist, only background cytoplasmic staining was observed in CREB−/− cells. These results demonstrate that CREB is indeed an important epitope for recognition by αK antibody. Under higher magnification, a punctate staining pattern was noted with αKK antiserum in forskolin stimulated D5 cells, suggesting that CREB:CBP complexes are formed in discrete loci within the nucleus.

### Example 5

### Fusion Complex

The nucleic acid sequence for the KID domain of CREB and the nucleic acid sequence for the KIX domain of CBP were subcloned into the pGEX4T3 vector in tandem. The two sequences were separated by a nucleic acid sequence which encodes a peptide having the sequence GSGPPSAKRPKLSSEFDIKLGTELGS (SEQ ID NO.: 13) and which corresponds to p300 nuclear localization signal (p300 NLS). The nucleic acid construct formed by the p300 NLS-linked KID/KIX fusion was inserted between the BamHI and Xma restriction sites of the pGEX4T3 expression vector. E. coli BL21Codon Plus cells harboring the vector were grown at 37° C. in LB media. The inducer IPTG (0.2 mM final concentration) was added to the culture for induction of target protein expression when cell growth reached an OD₆₀₀ of 1.0. The cells were harvested 4 hr after addition of IPTG by centrifugation. The cell pellet was resuspended in a buffer comprising 50 mM TRIS, pH 8.0, 150 mM NaCl and 1 mM PMSF. Cells were lysed by sonication and the resulting suspension was centrifuged. The cleared lysate was loaded onto a glutathione (GSH) affinity column. The protein was eluted using 10 mM reduced glutathione in 50 mM TRIS, pH 8.0. The gstKID-KIX protein was phosphorylated in vitro by incubating 26.4 μM purified protein with 540 nM protein kinase A (PKA) catalytic subunit in the presence of 300 μM ATP and 300 μM MgCl₂ in 25 mM TRIS, pH 7.0 at 30° C. for 1 hr. Phosphate incorporation was confirmed by Western blot. Previous NMR solution structure studies revealed that phospho-(Ser133)KID undergoes a conformational change from coil to helix upon binding to KIX. A polyclonal complex specific antiserum which detects residues in phospho-(Ser133)KID that undergo the conformational change following complex formation with KIX was able to detect the recombinant phospho-(Ser133)KID-KIX fusion in Western blot assays.

Compounds screened against phospho-(Ser133)KID and KIX individually can be screened against phospho-(SER)KID-KIX to produce compounds that will discriminate between the complex and either partner individually.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody that specifically recognizes a conformational epitope specific for a multimeric complex, wherein the antibody does not bind to any individual component of the multimeric complex when the individual component is not part of the multimeric complex.

2. The antibody of claim 1, wherein the antibody is a monoclonal antibody.

3. The antibody of claim 1, wherein the multimeric complex is a dimer.

4. The antibody of claim 3, wherein the multimeric complex is formed by the association of a cyclic AMP response element binding protein (CREB) and a CREB binding protein (CBP).

5. The antibody of claim 4, wherein the antibody binds at least one amino acid residue in a KIX domain of CBP, wherein the KIX domain undergoes a conformational change following formation of the multimeric complex.

6. The antibody of claim 4, wherein the antibody binds at least one amino acid residue in a kinase inducible domain (KID) of CREB that undergoes a conformational change following formation of the multimeric complex.

7. The antibody of claim 4, wherein the antibody recognizes lysine 136 of CREB.

8. The antibody of claim 4, wherein the antibody recognizes asparagine 139 of CREB.

9. The antibody of claim 1, wherein the antibody is αKK.

10. The antibody of claim 1, wherein the multimeric complex comprises a fusion protein.

11. The antibody of claim 10, wherein the fusion protein comprises CREB coupled to CBP.

12. The antibody of claim 10, wherein the fusion protein comprises a KID domain of CREB coupled to a KIX domain of CBP.

13. The antibody of claim 10, wherein the fusion protein comprises a first protein coupled to a second protein by a linker.

14. The antibody of claim 13, wherein the linker is flexible.

15. The antibody of claim 13, wherein the linker is cleavable.

16. The antibody of claim 13, wherein the linker is a chemical crosslinker.

17. The antibody of claim 13, wherein the linker is a peptide.

18. The antibody of claim 17, wherein the peptide is p300 nuclear localization signal (p300 NLS).

19. A method for identifying compositions specific for at least one component of a multimeric complex comprising:
providing at least one component of the multimeric complex and at least one candidate compound in a test solution;
contacting the test solution with an antibody that specifically recognizes a conformational epitope specific for the multimeric complex, wherein the antibody does not bind to any individual component of the multimeric complex when the individual component is not part of the multimeric complex; and
determining an effect of the at least one candidate compound on the binding of the multimeric complex to the antibody.

20. The method of claim 19, wherein the multimeric complex comprises CREB and CBP in physical communication.

21. The method of claim 20, wherein CREB is coupled to CBP thereby forming a fusion protein.

22. The method of claim 21, wherein CREB is coupled to CBP by a flexible linker.

23. The method of claim 20, wherein a KID domain of CREB is coupled to a KIX domain of CBP thereby forming a fusion protein.

24. The method of claim 20, wherein the effect of the at least one candidate compound on the binding of the CREB:CBP complex to the antibody is a blocking effect, wherein the blocking effect prevents binding of the antibody to the CREB:CBP complex.

25. The method of claim 20, wherein the blocking effect prevents formation of the CREB:CBP complex.

26. The method of claim 19, wherein the antibody is a monoclonal antibody.

27. The method of claim 19, wherein the effect recognizes a compound that disrupts the multimeric complex.

28. The method of claim 19, wherein the effect recognizes a compound that prevents the formation of the multimeric complex.

29. The method of claim 19, wherein the multimeric complex is a dimer.

30. The method of claim 29, wherein the multimeric complex is formed by the association of CREB and CBP, wherein said antibody does not bind to CREB or CBP when they are not part of said multimeric complex.

31. The method of claim 30, wherein the antibody binds at least one amino acid residue in a KIX domain of CBP, wherein the KIX domain undergoes a conformational change following formation of the multimeric complex.

32. The method of claim 30, wherein the antibody binds at least one amino acid residue in a KID domain that undergoes a conformational change following formation of the multimeric complex.

33. The method of claim 30, wherein the antibody recognizes lysine 136 of CREB.

34. The method of claim 30, wherein the antibody recognizes asparagine 139 of CREB.

35. The method of claim 19, wherein the antibody is αKK.

36. A method of treating an individual having a disorder resulting from the aberrant formation of a multimeric protein complex, said method comprising:
obtaining an antibody that specifically recognizes a conformational epitope specific for a multimeric complex, wherein the antibody does not bind to any individual component of the multimeric complex when the individual component is not part of the multimeric complex; and
administering said antibody in an amount sufficient to decrease the severity of the disorder.

37. The method of claim 36, wherein the multimeric complex is formed by the association of CREB and CBP.


SEQUENCE LISTING (13 sequences)

SEQ ID NO: 1 - protein, 5 aa, Artificial Sequence
  Linker
GGGGS

SEQ ID NO: 2 - protein, 12 aa, Artificial Sequence
  Linker
GKSSGSGSESKS

SEQ ID NO: 3 - protein, 14 aa, Artificial Sequence
  Linker
GSTSGSGKSSEGKG

SEQ ID NO: 4 - protein, 18 aa, Artificial Sequence
  Linker
GSTSGSGKSSEGSGSTKG

SEQ ID NO: 5 - protein, 18 aa, Artificial Sequence
  Linker
GSTSGSGKPGSGEGSTKG

SEQ ID NO: 6 - protein, 14 aa, Artificial Sequence
  Linker
EGKSSGSGSESKEF

SEQ ID NO: 7 - protein, 5 aa, Artificial Sequence
  Linker
SRSSG

SEQ ID NO: 8 - protein, 5 aa, Artificial Sequence
  Linker
SGSSC

SEQ ID NO: 9 - protein, 28 aa, Artificial Sequence
  Trypsin sensitive linker
AMGRSGGGCAGNRVGSSLSCGGLNLIAM

SEQ ID NO: 10 - protein, 22 aa, Caenorhabditis elegans
DEARRREQLNRRPSYRMILKDL

SEQ ID NO: 11 - protein, 22 aa, Mus musculus
DSHKRREILSRRPSYRKILNEL

SEQ ID NO: 12 - protein, 22 aa, Rattus norvegicus
DSQKRREILSRRPSYRKILNDL

SEQ ID NO: 13 - protein, 26 aa, Artificial Sequence
  p300 Nuclear localization signal
GSGPPSAKRPKLSSEFDIKLGTELGS